(19) United States
(12) Patent Application Publication
(10) Pub. No.: US 2020/0405649 A1
(43) Pub. Date: Dec. 31, 2020

(54) DNA ORIGAMI NANOPARTICLE DELIVERY OF PROGRAMMED CHROMOSOME BREAKAGE MACHINERY

(71) Applicant: RENSSELAER POLYTECHNIC INSTITUTE, Troy, NY (US)

(72) Inventors: Xing WANG, Cohoes, NY (US); John R. Bracht, Washington, DC (US)

(73) Assignee: RENSSELAER POLYTECHNIC INSTITUTE, Troy, NY (US)

(21) Appl. No.: 16/956,827

(22) PCT Filed: Dec. 21, 2018
(86) PCT No.: PCT/US18/67058

§ 371 Date: Jun. 22, 2020

Related U.S. Application Data
(60) Provisional application No. 62/609,938, filed on Dec. 22, 2017.

(51) Int. Cl. A61K 9/51; A61K 31/7105; C12N 15/11; A61K 38/46; C12N 9/22; A61K 48/00

(52) U.S. Cl. CPC A61K 9/513; A61K 9/5138; A61K 31/7105; C12N 15/11; C12N 2320/32; C12N 9/22; A61K 48/00; C12N 2310/20; A61K 38/465

(57) ABSTRACT

Compositions of the present disclosure include a first dCas9-FokI-sgRNA complex and a second dCas9-FokI-sgRNA complex. The first sgRNA is configured to target a first side of a genomic fusion site and the second sgRNA is configured to target a second side of the genomic fusion site. The first and second complexes only cut DNA upon dimerization. Thus, upon binding of both first dCas9-FokI-sgRNA complex and second dCas9-FokI-sgRNA complex, the dimer is produced and DNA cleavage proceeds at the genomic fusion site. The dCas9-FokI-sgRNA complexes are loaded on a folded-DNA shell for transport across the cellular membrane. The shell has a viral-mimic structure that maximizes cell entry, is non-cytotoxic, has low-to-nonimmunogenicity, and provides excellent capacity to enclose and protect the complexes. These systems exhibit both cellular (via the shell) and molecular (via the complexes) specificity, significantly reducing off-target activity and the associated harmful side-effects.

### CROSS REFERENCE TO RELATED APPLICATION(S)

This application is a national stage filing of International Patent Application No. PCT/US2018/067058, filed Dec. 21, 2018, which claims the benefit of U.S. Provisional Application No. 62/609,938, filed Dec. 22, 2017, which are incorporated by reference as if disclosed herein in their entirety.

### BACKGROUND

Chromosome fusion events are endemic to many cancers and in many cases are known drivers of oncogenic transformation. In perhaps the most famous example, Chronic myeloid leukemia (CML) is driven by a balanced translocation between chromosomes 9 and 22, creating the short “Philadelphia chromosome.” This translocation event fuses the ABL tyrosine kinase, a proto-oncogene on chromosome 9, with the ‘Breakpoint Cluster’ (BCR) region on chromosome 22, creating a novel kinase with aberrant growth and survival signaling activity. The development of the drug imatinib (Gleevec™, Novartis), a specific inhibitor of the BCR-ABL fusion protein, was a breakthrough in cancer treatment, converting CML from a fatal disease into a chronic treatable condition. With an overall response rate in clinical trials of 75% or higher, and few side effects, imatinib has been one of the great success stories in an ongoing struggle against cancer. However, approximately 25% of patients are imatinib-resistant, and some hospitals report substantially higher resistance levels. For these patients the list of treatment options is distressingly short and the need for additional treatments is urgent.

By way of further example, Acute Myeloid Leukemia (AML) very frequently contains a fusion translocation joining the AML1 and ETO genes, the resulting fusion being oncogenic. Another gene, MLL, can fuse with multiple partners to drive leukemia, accounting for the majority of childhood cases and 10% of adult-onset disease. Ewing's sarcoma, a bone cancer, is frequently driven by a promiscuous fusion of the EWS gene to multiple partner genes. This is apparently only the tip of the iceberg: one study used deep sequencing to discover over 9,000 additional cancer fusion genes. While the vast majority of these have been detected only once, and are likely not ‘driver’ mutations (i.e., not causing clonal expansion), in 330 cases recurrent gene fusions were found—potential drivers of malignancy.

Unfortunately, the success of imatinib remains an exception to the rule: most cancers do not have a corresponding drug targeting the fusion gene. Fusion genes are quite commonly identified in cancer genomes, but their functional role in cancer is unknown: are they mere ‘passenger’ events, occurring because of cancer's legendary genome instability, or are they ‘drivers’—pushing the transformation process forward?

### SUMMARY

Some embodiments of the present disclosure are directed to one or more protein-RNA complexes and a delivery system for transporting the protein-RNA complexes to a genomic fusion site. In some embodiments, a first protein-RNA complex includes one or more nucleases and a first sgRNA configured to target a first side of a genomic fusion site. In some embodiments, a second protein-RNA complex includes one or more nucleases and a second sgRNA configured to target a second side of the genomic fusion site. In some embodiments, the nucleases include FokI and deactivated Cas9. In some embodiments, the targets for the first sgRNA and the second sgRNA overlap a nuclease cleavage site. In some embodiments, a protospacer adjacent motif overlaps with the genomic fusion site.

In some embodiments, one or more protein-RNA complexes are loaded into or onto an ss-DNA shell for transport across the cellular membrane. In some embodiments, a homologous repair template is also loaded into or onto the ss-DNA shell for transport with the protein-RNA complexes. In some embodiments, a plurality of oligonucleotides are bound to the ss-DNA shell. In some embodiments, each of the oligonucleotides include a DNA extension configured to bind a protein-RNA complex. In some embodiments, the ss-DNA shell includes a shell coating configured to promote cellular uptake of the ss-DNA shell and thus the protein-RNA complexes. In some embodiments, the cellular coating includes aptamers for targeting a cell of interest. In some embodiments, the cellular coating includes a cationic polymer coating.

Some embodiments of the present disclosure are directed to a method of treating a patient having a genomic fusion translocation. In some embodiments, a genomic sample of the patient is obtained. In some embodiments, a genomic fusion site in the genomic sample is identified. In some embodiments, a first sgRNA binding site is identified at a first side of the genomic fusion site. In some embodiments, a second sgRNA binding site is identified at a second side of the genomic fusion site. In some embodiments, first and second protein-RNA complexes are prepared, the first protein-RNA complex having a first sgRNA configured to target the first sgRNA binding site and a second sgRNA configured to target the second sgRNA binding site. In some embodiments, the first protein-RNA complex and the second protein-RNA complex are administered to induce breakage of the genomic fusion site, e.g., to the patient to induce breakage in vivo or to the genomic sample to induce breakage in vitro. In some embodiments, the first and second protein-RNA complex are configured to cut DNA only upon dimerization. Thus, upon binding of both first protein-RNA complex and second protein-RNA complex at the genomic fusion site, the dimer is produced and DNA cleavage at the potentially oncogenic fusion site proceeds.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a schematic drawing of a composition for treating a patient with a genomic fusion translocation according to some embodiments of the present disclosure;

FIG. 1B is a schematic drawing of a composition for treating a patient with a genomic fusion translocation according to some embodiments of the present disclosure;

FIG. 1C is a schematic drawing of a composition for treating a patient with a genomic fusion translocation according to some embodiments of the present disclosure;

FIG. 2 is a schematic drawing of a protein-RNA complex delivery system according to some embodiments of the present disclosure;

FIG. 3A is a chart of a method for treating a patient having a genomic fusion translocation according to some embodiments of the present disclosure; and

FIG. 3B is a chart of a method for treating a patient having a genomic fusion translocation according to some embodiments of the present disclosure.

### DETAILED DESCRIPTION

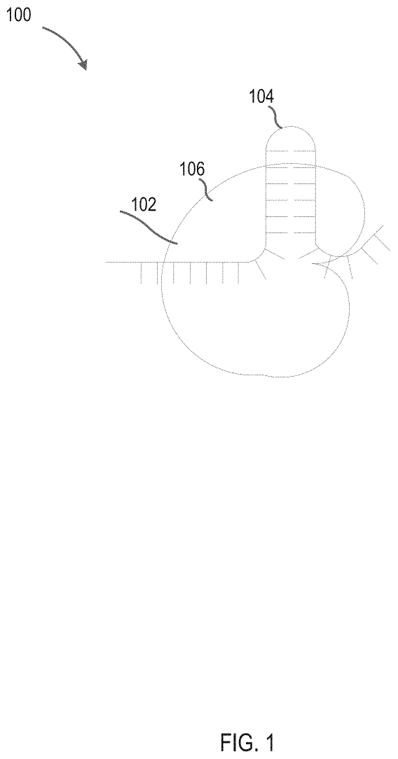

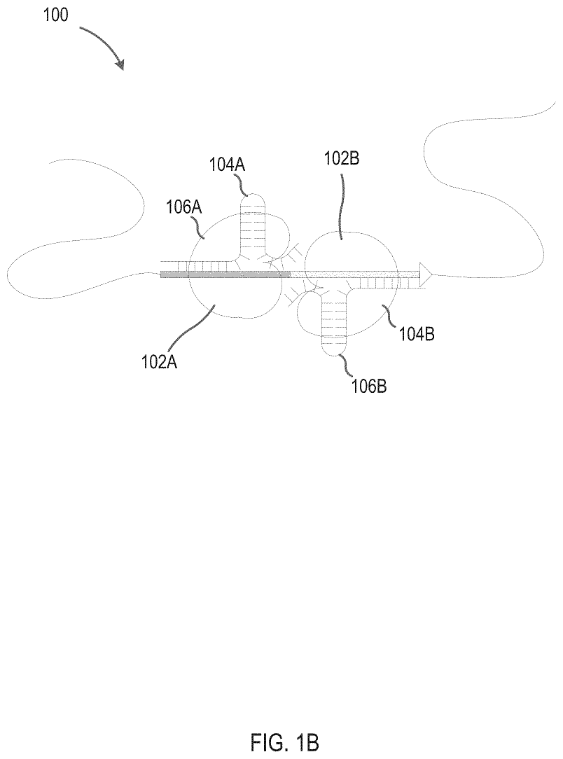

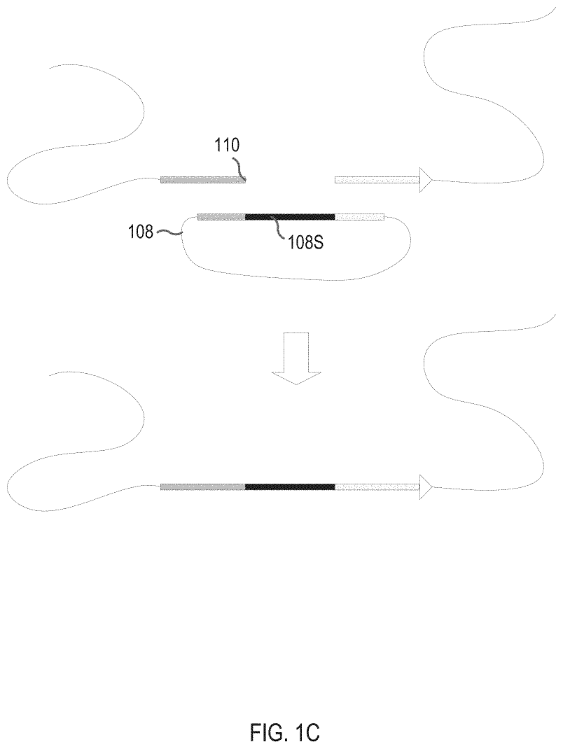

Referring now to FIG. 1A, some aspects of the disclosed subject matter include a composition 100 for treating a patient with a genomic fusion translocation. In some embodiments, the genomic fusion translocation is identified in at least one of the patient's cells. In some embodiments, the identification of the genomic fusion translocation is made in vivo. In some embodiments, the identification of the genomic fusion translocation is made in vitro. The translocation creates a fusion between genes in the patient genome at one or more genomic fusion sites. In some embodiments, the patient's genomic fusion translocation, and thus the resulting genomic fusion sites, are endemic of a cancer. In some embodiments, the patient's genomic fusion translocation, and thus the resulting genomic fusion sites, are drivers of the cancer's malignancy.

In some embodiments, composition 100 includes a protein-RNA complex 102 including a guide RNA (gRNA) 104. In some embodiments, gRNA 104 is a single guide RNA (sgRNA). In some embodiments, gRNA 104 is configured target the patient genome at a location proximate the genomic fusion site, e.g., has a sequence complementary to a location proximate the genomic fusion site. In some embodiments, gRNA 104 is configured to target the patient genome at a location that at least partially overlaps the genomic fusion site. In some embodiments, protein-RNA complex 102 includes one or more nucleases 106. In some embodiments, protein-RNA complex 102 includes FokI and deactivated Cas9. In some embodiments, protein-RNA complex 102 is a nickase. In some embodiments, one or more nucleases 106 are configured to bind a DNA strand at a protospacer adjacent motif (PAM). In some embodiments, the PAM overlaps with the genomic fusion site.

Protein-RNA complex 102 is configured to identify a target sequence in a DNA strand and cleave, via the one or more nucleases, the DNA at a specific cleavage site. In some embodiments, the identification and cleavage of the DNA strand by protein-RNA complex 102 occurs in vivo, e.g., in a nucleus of a patient's cell. In some embodiments, the identification and cleavage of the DNA strand by protein-RNA complex 102 occurs in vitro, e.g., as a test on a sample of patient cells removed from the patient and analyzed in a lab. Referring now to FIG. 1B, in some embodiments, composition 100 includes a plurality of protein-RNA complexes 102. In some embodiments, the plurality of protein-RNA complexes 102 include two or more different gRNA 104. In some embodiments, composition 100 includes a first protein-RNA complex 102A and a second protein-RNA complex 102B. In some embodiments, first protein-RNA complex 102A includes a first gRNA 104A configured to target a first side of the genomic fusion site. In some embodiments, second protein-RNA complex 102B includes a second gRNA 104B configured to target a second side of the genomic fusion site. In some embodiments, as discussed above, plurality of protein-RNA complexes 102 include one or more nucleases. In some embodiments, first gRNA 104A overlaps the nuclease cleavage site. In some embodiments, second gRNA 104B overlaps the nuclease cleavage site. In some embodiments, both first gRNA 104A and second gRNA 104B overlap the nuclease cleavage site. In these embodiments, the genomic fusion site becomes bracketed by protein-RNA complexes 102, with a first protein-RNA complex 102A being guided by first gRNA 104A to a first side of the fusion site and a second protein-RNA complex 102B being guided by second gRNA 104B to a second side of the fusion site. In embodiments wherein protein-RNA complexes 102 include FokI and deactivated Cas9, the complex is reliant upon dimerization to actually cleave the DNA. If only one of first protein-RNA complex 102A or second protein-RNA complex 102B are bound proximate the genomic fusion site without the other, dimerization does not occur and the DNA will not be cleaved. However, upon binding of both first protein-RNA complex 102A and second protein-RNA complex 102B, the dimer is produced and DNA cleavage proceeds. Because the genomic fusion sites are essentially specific to cancerous or otherwise affected cells, and further because of the specificity of the gRNAs for identified sites proximate to the fusion sites, healthy cells and healthy genome within affected cells will fail to properly bind first protein-RNA complex 102A or second protein-RNA complex 102B and thus be left unaffected.

Referring now to FIG. 1C, in some embodiments, composition 100 includes a homologous repair template 108. Homologous repair template 108 is configured to repair the DNA break 110 created by protein-RNA complex 102. In some embodiments, homologous repair template 108 includes one or more homology arms. In some embodiments, homologous repair template 108 includes a polynucleotide sequence encoding an insertion construct 108S for subsequent expression by the cell, e.g., as a gene drive. In some embodiments, the insertion construct encodes the protein-RNA complex 102 itself.

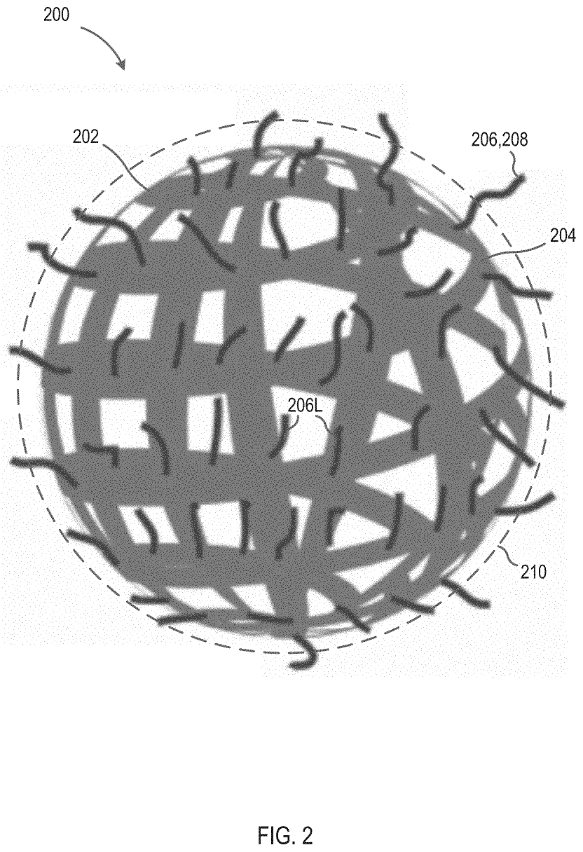

Referring now to FIG. 2, in some embodiments, protein-RNA complex 102 is transported into a patient cell of interest via a delivery system 200. In some embodiments, delivery system 200 includes a shell 202 configured to hold one or more protein-RNA complexes 102 and facilitate transportation of composition 100 across a cellular membrane. In some embodiments, shell 202 is configured to facilitate transportation of composition 100 across a cellular membrane and into an endosome. In some embodiments, shell 202 is configured to facilitate transportation of composition 100 across a cellular membrane and out of an endosome. In some embodiments, shell 202 is configured to facilitate transportation of composition 100 across a cellular membrane and a nuclear membrane. In some embodiments, shell 202 is also configured to release composition 100 within the patient cell of interest, where the composition can subsequently be guided to a genomic fusion site. In some embodiments, shell 202 has a diameter of about 10, about 20, about 30, about 40, or about 50 nm. In some embodiments, shell 202 further includes homologous repair template 108.

In some embodiments, a plurality of shells 202 holding one or more protein-RNA complexes 102, i.e., composition 100, form a therapeutic medicament, e.g., for treatment of an illness such as cancer in a patient. In some embodiments, a plurality of shells 202 holding one or more protein-RNA complexes 102, i.e., composition 100, are a part of a diagnostic test kit for identifying genomic fusion sites. In each of these embodiments, the size distribution of shells 202 in a unit of composition 100 is substantially uniform.

In some embodiments, shell 202 is composed of single stranded DNA (ss-DNA). In some embodiments, shell 202 is composed of a combination of ss-DNA and double-helical DNA. In some embodiments, shell 202 is generally spherical, hemispherical, or any other suitable shape, or combinations thereof. In some embodiments, shell 202 is hollow. In some embodiments, shell 202 is composed of two hemispheres connected equatorially to create a spherical shell. In some embodiments, shell 202 includes a plurality of latitudinal rings 204. In some embodiments, the latitudinal rings are composed of ss-DNA.

In some embodiments, a plurality of oligonucleotides 206 are bound to shell 202. In some embodiments, oligonucleotides 206 promote self-assembly of shell 202 into the desired shape. In some embodiments, the number is oligonucleotides 206 bound to shell 202 is greater than about 50. In some embodiments, the number is oligonucleotides 206 bound to shell 202 is greater than about 100. In some embodiments, oligonucleotides 206 include a DNA extension 208. In some embodiments, DNA extension 208 is an ss-DNA attached to the 3′-end of the oligonucleotides. In some embodiments, DNA extension 208 is configured to reversibly bind protein-RNA complex 102. In some embodiments, DNA extension 208 binds gRNA 106 in protein-RNA complex 102. In some embodiments, oligonucleotides 206 are disposed on the outside of the shell 202, the inside of shell 202, or combinations thereof. In some embodiments, delivery system 200 is prepared via mixing the ss-DNA with oligonucleotides 206 in an appropriate buffer, e.g., Mg²⁺ buffer, and subject the mixture to a thermal annealing process, e.g., from about 90° C. to about 4° C. over 24 hours, resulting in self-assembly of shells 202. Composition 100 is then mixed with shells 202 to load delivery system 200.

In some embodiments, e.g., where shell 202 is composed of two hemispheres, shell 202 includes a plurality of locking staple oligonucleotide strands 206L. Strands 206L are configured to reversibly combine a first hemispherical shell with a second hemispherical shell to create the spherical shell.

In some embodiments, shell 202 includes a shell coating 210. In some embodiments, shell coating 210 is configured to promote cellular uptake of shell 202. In some embodiments, shell coating 210 includes a plurality of target-cell aptamers bound to shell 202. Use of aptamers further increases the specificity of composition 100 and delivery system 200. These aptamers enable delivery system 200 to target only those cells of interest. Thus, composition 100 is only delivered to where it is needed, further minimizing the chance of genomic damage to otherwise healthy cells. In some embodiments, shell coating 210 includes a cationic polymer, e.g., polyethylenimine.

In some embodiments, delivery system 200 includes a lipid nanoparticle based delivery platform (not pictured). In some embodiments, the delivery system includes dendrimers, chitosan, gold nanoparticles, or combinations thereof (not pictured).

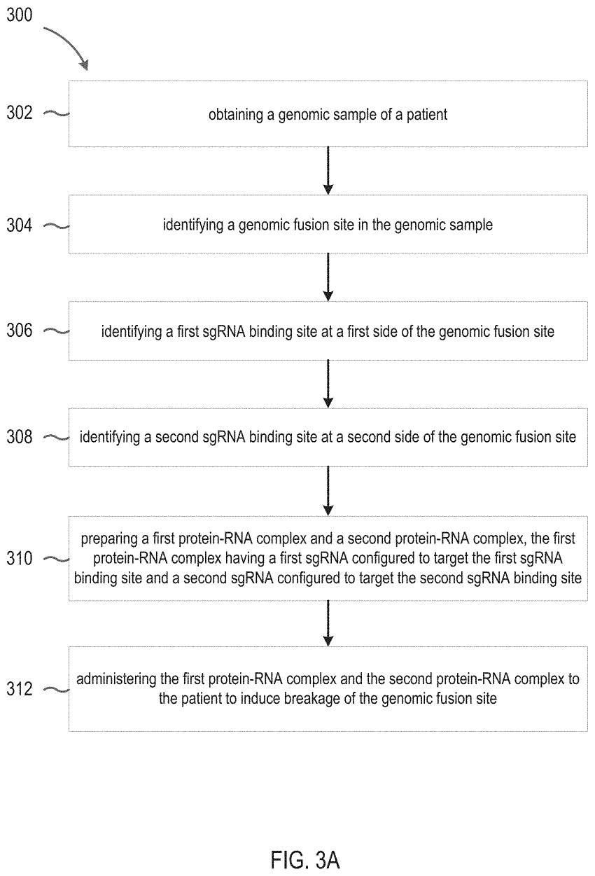

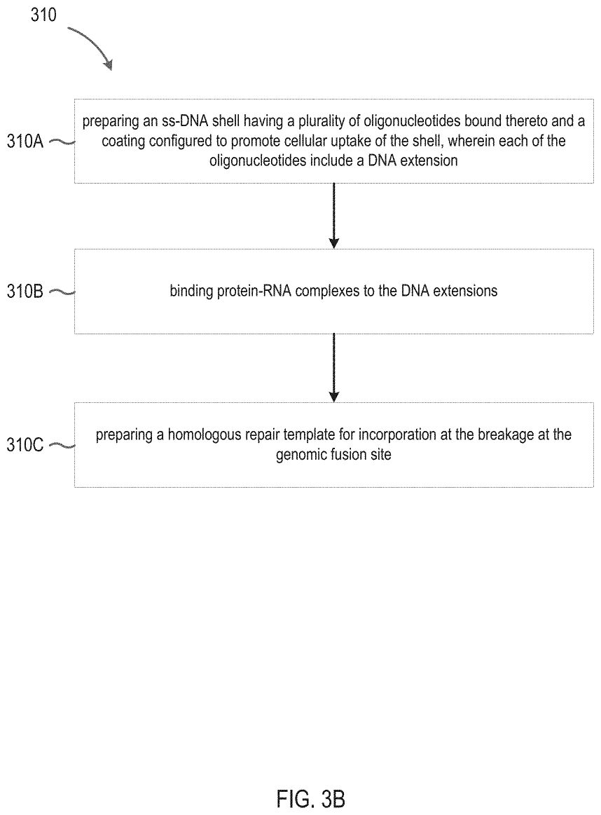

Referring now to FIG. 3A, some embodiments of the present disclosure are directed to a method 300 of treating a patient having a genomic fusion translocation. In some embodiments, at 302, a genomic sample of the patient is obtained. At 304, a genomic fusion site in the genomic sample is identified. At 306, a first gRNA binding site at a first side of the genomic fusion site is identified. At 308, a second sgRNA binding site at a second side of the genomic fusion site is identified. At 310, first protein-RNA and second protein-RNA complexes are prepared. As discussed above, the first protein-RNA complex has a first gRNA configured to target the first gRNA binding site and a second gRNA configured to target the second gRNA binding site. At 312, the first and second protein-RNA complexes are administered to the patient to induce breakage of the genomic fusion site, e.g., by one or more nucleases.

Referring now to FIG. 3B, in some embodiments, preparing 310 the first and second protein-RNA complexes includes preparing 310A a delivery shell. As discussed above, in some embodiments, the shell includes a plurality of oligonucleotides (including DNA extensions) bound thereto and a coating configured to promote cellular uptake of the shell. At 310B, protein-RNA complexes are bound to the DNA extensions for transportation towards the genomic fusion site. At 310C, a homologous repair template is prepared for incorporation at the breakage at the genomic fusion site.

Methods and systems of the present disclosure advantageously provide a DNA origami delivery system with a viral-mimic structure that maximizes cell entry; is non-cytotoxic (in contrast to gold particles), low-to-nonimmunogenic (in contrast to viral packaging), and provides excellent capacity to enclose and protect the payload through the rigors of the in vivo environment. These benefits are significant for both clinical work, e.g., treatments for cancer patients, laboratory work, e.g., assessment of functional roles of chromosome fusions in cancer, and drug delivery research, e.g., other payloads being coupled to the nanoparticles delivery.

The delivery system serves as a ‘viral capsid-analog’ platform decorated with targeting aptamers or coated with polymers, yet is non-viral in sequence, thus avoiding immunogenicity; at the same time the particle sizes are both controllable and uniform like gold nanoparticles while avoiding the cytotoxicity they engender and providing better payload protection and precision attachment of functional groups. DNA based system also provides precise functionalization (e.g. placement of protein, aptamer, and small drug ligands with nanometer-scale accuracy), along with better cellular targeting by specific aptamers or other active molecules. This improved specificity is also enjoyed by the polymer-RNA complexes, which by activating only in tandem greatly limit off-target binding and cleavage of patient DNA. Finally, the ability to specifically cleave and repair genomic fusion sites opens up a variety of diagnostic and therapeutic possibilities to aid those battling illness caused by fusion translocations.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A protein-RNA complex delivery system comprising:
an ss-DNA shell;
a plurality of oligonucleotides bound to the ss-DNA shell, wherein each of the oligonucleotides include a DNA extension configured to bind a protein-RNA complex; and
a shell coating configured to promote cellular uptake of the ss-DNA shell.

2. The system according to claim 1, wherein the ss-DNA shell is spherical, hemispherical, or combinations thereof.

3. The system according to claim 2, further comprising a plurality of locking staple oligonucleotide strands configured to combine a first ss-DNA hemisphere with a second ss-DNA hemisphere into an ss-DNA sphere.

4. The system according to claim 3, wherein the plurality of oligonucleotides are bound to the inner surface of the ss-DNA shell.

5. The system according to claim 1, wherein the system includes one or more protein-RNA complexes reversibly bound to the ss-DNA shell, each of the protein-RNA complexes including one or more nucleases and an sgRNA.

6. The system according to claim 5, wherein the system includes a first protein-RNA complex and a second protein-RNA complex, the first protein-RNA complex having a first sgRNA configured to target a first side of a genomic fusion site identified in a patient cell and a second sgRNA configured to target a second side of the genomic fusion site.

7. The system according to claim 6, wherein the protein-RNA complex includes FokI and deactivated Cas9.

8. The system according to claim 6, wherein the targets for first sgRNA and the second sgRNA overlap a nuclease cleavage site.

9. The system according to claim 6, further comprising a protospacer adjacent motif overlapping with the genomic fusion site.

10. The system according to claim 1, wherein the shell coating includes a plurality of target-cell aptamers bound to the ss-DNA shell.

11. The system according to claim 1, wherein the shell coating includes a cationic polymer.

12. The system according to claim 11, wherein the cationic polymer includes polyethylenimine.

13. The system according to claim 1, further comprising a homologous repair template.

14. A method of treating a patient having a genomic fusion translocation, the method comprising:
obtaining a genomic sample of a patient;
identifying a genomic fusion site in the genomic sample;
identifying a first sgRNA binding site at a first side of the genomic fusion site;
identifying a second sgRNA binding site at a second side of the genomic fusion site;
preparing a first protein-RNA complex and a second protein-RNA complex, the first protein-RNA complex having a first sgRNA configured to target the first sgRNA binding site and a second sgRNA configured to target the second sgRNA binding site; and
administering the first protein-RNA complex and the second protein-RNA complex to the patient to induce breakage of the genomic fusion site;
wherein the first protein-RNA complex and a second protein-RNA complex include one or more nucleases.

15. The method according to claim 14, wherein the protein-RNA complex includes FokI and deactivated Cas9.

16. The method according to claim 14, wherein preparing the first protein-RNA complex and the second protein-RNA complex further comprises:
preparing an ss-DNA shell having a plurality of oligonucleotides bound thereto and a coating configured to promote cellular uptake of the shell, wherein each of the oligonucleotides include a DNA extension; and
binding protein-RNA complexes to the DNA extensions.

17. The method according to claim 14, wherein preparing the first protein-RNA complex and the second protein-RNA complex further comprises:
preparing a homologous repair template for incorporation at the breakage at the genomic fusion site.

18. A composition for treating a patient with a genomic fusion translocation, the composition comprising:
a first protein-RNA complex including one or more nucleases and a first sgRNA configured to target a first side of a genomic fusion site identified in a patient cell; and
a second protein-RNA complex including one or more nucleases and a second sgRNA configured to target a second side of the genomic fusion site;
wherein the protein-RNA complex includes FokI and deactivated Cas9.

19. The system according to claim 18, wherein the first sgRNA and the second sgRNA overlap a nuclease cleavage site.

20. The system according to claim 18, further comprising a homologous repair template.
